(19) United States
(12) Patent Application Publication
(10) Pub. No.: US 2025/0114591 A1
(43) Pub. Date: Apr. 10, 2025

(54) BLOOD PUMP SYSTEMS AND METHODS

(71) Applicant: Shifamed Holdings, LLC, Campbell, CA (US)

(72) Inventors: Ari RYAN, Sunnyvale, CA (US); Jonelle BALDUCCI, Campbell, CA (US); Daniel HILDEBRAND, Santa Cruz, CA (US); Seyed Mostafa GHOREYSHI, Campbell, CA (US)

(21) Appl. No.: 18/833,857

(22) PCT Filed: Jan. 30, 2023
(86) PCT No.: PCT/US2023/061579

§ 371 Date: Jul. 26, 2024

Related U.S. Application Data
(60) Provisional application No. 63/267,286, filed on Jan. 28, 2022.

(51) Int. Cl. A61M 60/17; A61M 60/216; A61M 60/408; A61M 60/857

(52) U.S. Cl. CPC A61M 60/17; A61M 60/216; A61M 60/408; A61M 60/857; A61M 2207/10

(57) ABSTRACT

Catheter blood pumps that include an expandable conduit coupled to at least one hub shaped to promote smooth blood flow. In some examples, the at least one hub includes non-metallic configuration and the struts comprise a metallic configuration (e.g., nitinol). The hub can include one or more layers, including a combination of Chronoflex and Pebax.

### CROSS REFERENCE TO RELATED APPLICATIONS

This application claims the benefit of priority to U.S. Application No. 63/267,286, filed Jan. 28, 2022.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. Intra-aortic balloon pumps (IABP) are used to support circulatory function, such as treating heart failure patients. An IABP is typically placed within the aorta, and inflated and deflated in counter-pulsation fashion with the heart contractions, with one function being to provide additive support to the circulatory system. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

More recently, minimally invasive rotary blood pumps have been developed, which are inserted into the body in connection with the cardiovascular system to pump arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient. The smallest rotary blood pumps currently available may be percutaneously inserted into the vasculature of a patient through an access sheath, thereby avoiding more extensive surgical intervention, or through a vascular access graft. One such device is a percutaneously inserted ventricular support device.

Although blood pumps exist, there is a need to provide additional improvements in the field of ventricular support devices and similar blood pumps for treating compromised cardiac blood flow.

### SUMMARY OF THE DISCLOSURE

An intravascular blood pump is provided, comprising a collapsible conduit having an inner lumen for passing fluid therethrough, the conduit comprising a proximal end having a proximal opening, and a distal end having a distal opening, at least one impeller within the conduit, the at least one impeller arranged to pump fluid into the distal opening of the conduit and out of the proximal opening of the conduit, a plurality of metallic struts extending from the proximal end or the distal end of the conduit, and a hub configured to receive the plurality of metallic struts, the hub comprising at least one non-metallic layer.

In some embodiments, the hub comprises a first non-metallic layer and a second non-metallic layer disposed around the plurality of metallic struts.

In one embodiment, the first non-metallic layer comprises a thermoplastic urethane.

In some examples, the second non-metallic layer comprises Pebax.

In some embodiments, the at least one non-metallic layer is configured to encompass or surround the plurality of metallic struts.

In other embodiments, the first non-metallic layer and the second non-metallic layer are heat treated so as to meld together around the plurality of metallic struts.

In one example, the hub comprises a distal hub positioned adjacent to the distal opening.

In some embodiments, the blood pump further comprises a bullet-shaped tapering section extending proximally from where the plurality of metallic struts are joined to the distal hub.

In some examples, the plurality of metallic struts comprise nitinol.

In some embodiments, the plurality of metallic struts comprises 4, 5, 6, 7, or 8 struts.

An intravascular blood pump is provided, comprising a collapsible conduit having an inner lumen for passing fluid therethrough, the conduit comprising a proximal end having a proximal opening, and a distal end having a distal opening, a proximal impeller positioned at least partially within the conduit near the proximal opening, the at least one impeller arranged to pump fluid into the distal opening of the conduit and out of the proximal opening of the conduit, a plurality of metallic struts extending from the distal end of the conduit, and a distal hub configured to receive the plurality of metallic struts, the hub comprising at least one non-metallic layer configured to encapsulate the plurality of metallic struts.

In some embodiments, the hub comprises a first non-metallic layer and a second non-metallic layer disposed around the plurality of metallic struts.

In one embodiment, the first non-metallic layer comprises a thermoplastic urethane.

In some examples, the second non-metallic layer comprises Pebax.

In some embodiments, the at least one non-metallic layer is configured to encompass or surround the plurality of metallic struts.

In other embodiments, the first non-metallic layer and the second non-metallic layer are heat treated so as to meld together around the plurality of metallic struts.

In one example, the hub comprises a distal hub positioned adjacent to the distal opening.

In some embodiments, the blood pump further comprises a bullet-shaped tapering section extending proximally from where the plurality of metallic struts are joined to the distal hub.

In some examples, the plurality of metallic struts comprise nitinol.

In some embodiments, the plurality of metallic struts comprises 4, 5, 6, 7, or 8 struts.

A method of manufacturing an intravascular blood pump, the method comprising placing one or more metallic struts of a blood conduit on or near a first non-metallic hub layer, placing a second non-metallic hub layer on or over the one or more metallic struts, and applying a heat treatment to the one or more metallic struts, the first non-metallic hub layer, and the second non-metallic hub layer to meld or melt the first and second non-metallic hub layers around the one or more metallic struts.

In some embodiments, prior to the applying a heat treatment step, applying a shrink tubing over the second non-metallic hub layer.

In some implementations, the method can further include removing the shrink tubing after applying the heat treatment.

### BRIEF DESCRIPTION OF THE DRAWINGS

FIG. 1 is a side view of an exemplary blood pump that includes an expandable scaffold that supports a blood conduit with an impeller housed therein.

FIGS. 2A-2C illustrate one embodiment of a hub assembly of a blood pump.

FIG. 3 is a flowchart indicating an exemplary method of using a blood pump.

### DETAILED DESCRIPTION

The present disclosure is related to medical devices, systems, and methods of use and manufacture. In particular, described herein are pumps adapted to be disposed within a physiologic vessel, wherein the distal pump portion includes one or more components that act upon fluid. For example, the pumps herein may include one or more rotating members that when rotated, can facilitate the movement of a fluid such as blood.

Any of the disclosure herein relating to an aspect of a system, device, or method of use can be incorporated with any other suitable disclosure herein. For example, a figure describing only one aspect of a device or method can be included with other embodiments even if that is not specifically stated in a description of one or both parts of the disclosure. It is thus understood that combinations of different portions of this disclosure are included herein.

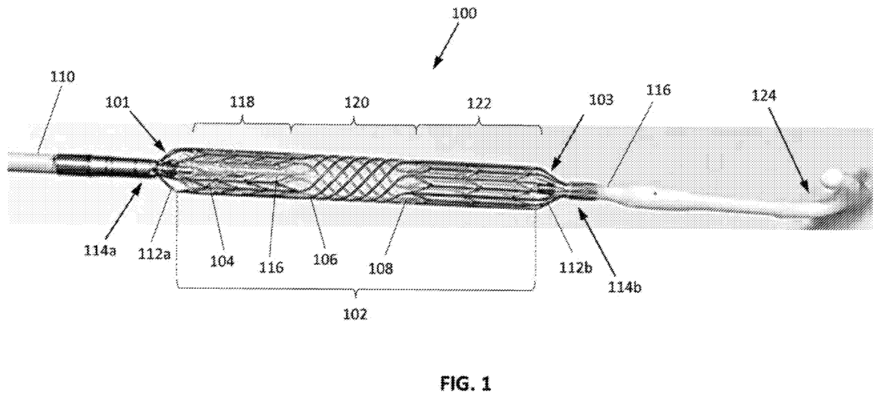

FIG. 1 shows a side view of one embodiment of an intravascular catheter blood pump 100. The blood pump 100 includes an expandable/collapsible blood conduit 102 that is configured to transition between an expanded state, as shown in FIG. 1, and a collapsed state (not shown). For example, the conduit 102 may be in the collapsed state when confined within a delivery catheter for delivery to the heart, expanded upon release from the delivery catheter for blood pumping, and collapsed back down within the delivery catheter (or other catheter) for removal from heart. When in the expanded state, the conduit 102 is radially expanded so as to form an inner lumen for passing blood therethrough. In some embodiments, the conduit 102 is impermeable to blood. When in the expanded state, the inner lumen of the conduit 102 may be configured to accommodate blood pumped by one or more impellers therein. The one or more impellers may be collapsible so that they may collapse to a smaller diameter when the conduit 102 is in the collapsed state. The one or more impellers may be positioned within one or more impeller regions of the conduit 102. In some examples, the impeller region(s) of the conduit 102 is/are radially stiffer than other regions (e.g., adjacent regions) of the conduit 102 to prevent the impeller(s) from contacting the interior walls of the conduit 102.

In this example, the blood pump 100 includes an impeller 104 within a proximal portion of the conduit 102. In some cases, the blood pump 100 can include more than one impeller. For example, the blood pump 100 may include a second impeller in a distal region 122 of the fluid conduit 102. In some cases, blood pump 100 may include more than two impellers. The conduit 102 includes a first (e.g., proximal) end having a first (e.g., proximal) opening 101, and a second (e.g., distal) end having a second (e.g., distal) opening 103. The first opening 101 and second opening 103 may be configured as and an inlet and outlet for blood. For example, blood may largely enter the conduit 102 via the second (e.g., distal) opening 103 and exit the conduit 102 via the first (e.g., proximal) opening 101. In such case, the second opening 103 acts as a blood inlet and the first opening 101 acts as a blood outlet. The one or more impellers (e.g., impeller 104) may be configured to pump blood from the inlet toward the outlet. In an exemplary operating position, the second opening 103 (e.g., inlet) may be distal to the aortic valve, in the left ventricle, and the first opening 101 (e.g., outlet) may be proximal to the aortic valve (e.g., in the ascending aorta).

The conduit 102 can include a tubular expandable/collapsible scaffold 106 that provides structural support for a membrane 108 that covers at least a portion of inner surfaces and/or outer surfaces of the scaffold 106. The scaffold 106 includes a material having a pattern of openings with the membrane 108 covering the openings to retain the blood within the lumen of the conduit 102. The scaffold 106 may be unitary and may be made of a single piece of material. For example, the scaffold 106 may be formed by cutting (e.g., laser cutting) a tubular shaped material. Exemplary materials for the scaffold 106 may include one or more of: nitinol, cobalt alloys, and polymers, although other materials may be used.

The blood pump 100 can further include proximal struts 112a that extend from the scaffold 106 near the first opening 101 (e.g., blood outlet region) and distal struts 112b that extend from the scaffold 106 near the second opening 103 (e.g., blood inlet region). The proximal struts 112a are coupled to first hub 114a of a proximal shaft 110. The distal struts 112b are coupled to second hub 114b of a distal portion 114. In this example, the first hub 114a includes a bearing assembly through which a central drive cable 116 extends. The drive cable 116 is operationally coupled to and configured to rotate the impeller 104.

In some cases, the impeller 104 is fully positioned axially within the conduit 102. In other cases, a proximal portion of the impeller 104 is positioned at least partially outside of the conduit 102. That is, at least a portion of the impeller may be positioned in axially alignment with a distal portion of the struts 112a.

The conduit 102 and the scaffold 106 may characterized as having a proximal region 118, a central region 120, and a distal region 122. The central region 120 may be configured to be placed across a valve (e.g., aortic valve) such that the proximal region 118 is at least partially within a first heart region (e.g., ascending aorta) and the distal region 122 is at least partially within a second heart region (e.g., left ventricle). In some embodiments, the central portion may be more flexible than the proximal and distal regions. The proximal region 118 (and in some cases the distal region 122) may be configured to house an impeller therein. The proximal region 118 may (and in some cases the distal region 122) has a stiffness sufficient to withstand deformation during operation of the blood pump 100 when within the beating heart and to maintain clearance (i.e., a gap) between an impeller region of the blood pump 100 and the rotating impeller 104. The distal region 122 includes the second (e.g., distal) opening 103 of the conduit 102, and may serve as the blood inlet for the conduit 102.

The central region 120 may be less rigid relative to the proximal region 118 (and in some cases the distal region 122). The higher flexibility of the central region 120 may allow the central region 120 to deflect when a lateral force is applied on a side of the conduit 102, for example, as the conduit 102 traverses through the patient's blood vessels and/or within the heart. For example, the central region 120 may be configured to laterally bend upon a lateral force applied to the distal region 122 and/or the proximal region 118. In some cases, it may be desirable for the central region 120 to laterally bend as the conduit 102 traverses the ascending aorta and temporarily assume a bent configuration when the conduit 102 is positioned across an aortic valve. In this example, the central region 120 includes a helical arrangement of longitudinally running elongate elements configured to provide flexibility for lateral bending. In some examples, a distal tip 124 of the blood pump 100 is curved to form an atraumatic tip. In some cases, the distal tip 124 flexible (e.g., laterally bendable) to enhance the atraumatic aspects of the distal tip 124. For example, the distal tip 124 may be sufficiently flexible to bend when pressed against tissue (e.g., by a predetermined amount of force) to prevent puncture of the tissue.

The first hub 114a (e.g., proximal hub) and/or the second hub 114b (e.g., distal hub) may include features that promote smooth blood flow into and/or out of the conduit 102 and/or are configured to reduce areas of stagnant flow that may promote clotting or thrombosis. The first and second hubs may further include features for attaching or connecting to the struts, scaffold, and/or conduit of the blood pump. Such features may prevent or reduce the occurrence of stagnant and/or turbulent blood flow that may otherwise tend to occur in regions near the first opening 101 (e.g., outlet region) and/or the second opening 103 (e.g., inlet region) of the conduit 102. Since stagnant and/or turbulent blood flow is associated with blood coagulation and/or clotting, measures to reduce this can be beneficial to for patient outcome.

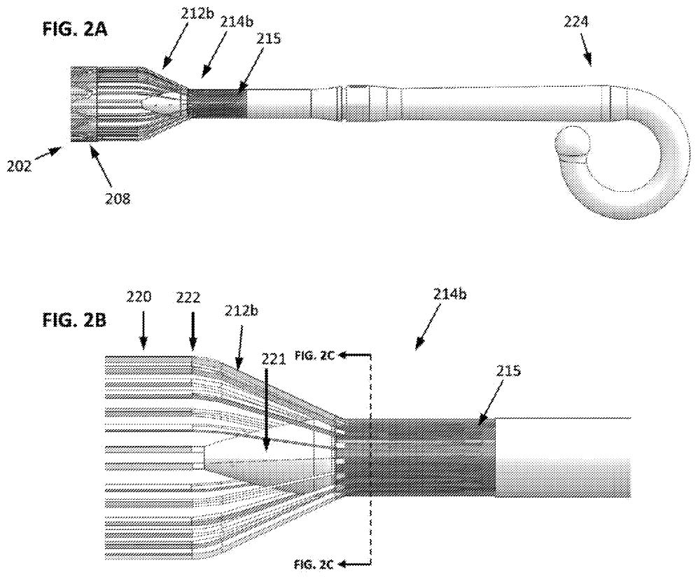

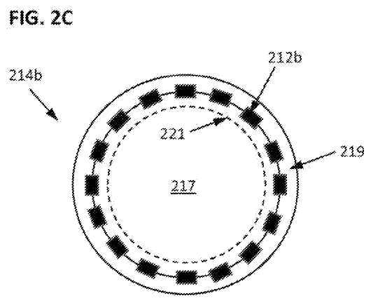

FIG. 2A-2B illustrate components of a blood pump with an exemplary distal hub 214b configured to attach to the struts 212b of the blood conduit and further configured to promote non-turbulent fluid flow past/through the hub and struts and into the conduit. Additionally, the configuration of the distal hub and its attachment to the struts can be designed and configured to prevent, reduce, or limit areas of stagnant blood flow, particularly under the distal struts of the scaffold, to prevent, reduce, or limit clot formation at or near the hub or struts. FIG. 2A shows a side view of a distal end of a conduit 202, which includes a membrane 208 covering a portion of struts (e.g., distal struts) 212b that extend from the distal end of the conduit 202. FIG. 2A further shows a distal tip 224 of the blood pump that can be curved to form an atraumatic tip.

FIG. 2B further shows a close up view of an attachment region 215 of the hub 214b. This attachment region 215 defines the location in the hub 214b where the struts 212b are connected or attached to the hub. Any number of struts 212b (e.g., 1, 2, 3, 4, 5, 6, 7, 8, 9, 10, 11 or more, etc.) can be implemented in the blood pump and attached to the hub. In some exemplary embodiments, the blood pump includes only 4, 5, 6, 7, or 8 struts.

In the embodiment of FIG. 2B, the hub region can comprise a non-metallic material, such as a urethane or a polyurethane. In contrast, the struts can comprise a metallic or shape memory material, such as nitinol. In one embodiment, the metallic struts can be surrounded, encased, or encompassed by the non-metallic hub material(s). The struts can be encased or encapsulated between two or more layers of a non-metallic material. For example, a first non-metallic material can be positioned on an inside of the struts and a second non-metallic material can be positioned on an outside of the struts.

FIG. 2B further shows a bullet-shaped extending portion 221 that gradually tapers away proximally from where the struts attach or connect to the hub. This tapered portion can include a taper/slope away from the struts configured to promote smooth blood flow past the struts and further configured to resist or prevent stagnant blood flow, particularly in the regions of the extending portion 221 that are underneath or near the struts 212b. It can be seen that the extending portion 221 has a first diameter at the hub that is larger than a second diameter at a proximal end of the extending portion. In some embodiments, the proximal end can be flat, as shown. In other embodiments, the proximal end can taper down to a point, or alternatively, can have a curved surface.

In some embodiments, the extending portion 221 can extend proximally from the distal hub 214b towards the blood conduit. As shown in FIG. 2B, the extending portion 221 has a distal end positioned at the hub 214b and a proximal end positioned just distally to interface 222 where the struts 212b meet the scaffold 220 of the blood conduit. In some embodiments, the proximal end of the extending portion 221 may extend proximally into the scaffold 220 of the blood conduit. In some embodiments, the struts 212b are integral with the scaffold 220, and the interface 222 is simply where the struts stop tapering outwards and begin to extend generally parallel along the length of the blood conduit.

FIG. 2C is a cross sectional view of the embodiment of FIG. 2C, showing one specific embodiment for the attachment of the struts 212b to the distal hub 214b. In this embodiment, the struts 212b can be sandwiched, encased, or encompassed by two or more urethane layers 217 and 219. In one embodiment, the first layer 217 can comprise a central core and the struts 212 can be placed on top of the first layer. The second layer 219 can then be placed over the first layer and the struts to encompass the struts in non-metallic material. In another embodiment, the first layer 217 need not be a central core, but instead can be a hollow layer as indicated by dashed line 221.

In some embodiments, heat treatment or other manufacturing methods can be applied to the strut/hub/urethane assembly to melt and or mold the various urethane layers into and around the struts. For example, in one specific embodiment the first layer can be a polycarbonate urethane layer (e.g., Chronoflex) and the second layer can be a urethane and nylon polymer (e.g., Pebax). Heat treatment can be applied to cause the first layer and the second layer to flow/melt together in and around the struts. In one specific embodiment, a shrink tubing layer or sleeve can be placed over the first layer, second layer, and struts prior to heat treatment. When heat treatment is applied to the assembly, the shrink tubing layer or sleeve can help or aid the urethane layers to melt or merge in a more uniform manner. In some examples, the shrink tubing layer or sleeve can be removed after heat treatment.

Encompassing the struts in a non-metallic material, such as is described herein, can advantageously eliminate rough or sharp edges defined by the struts, which can result in more even or less turbulent blood flow past the struts during use of the pump. As a result, clotting and/or stagnation can be prevented or limited, decreasing the chance of blood clots forming during use.

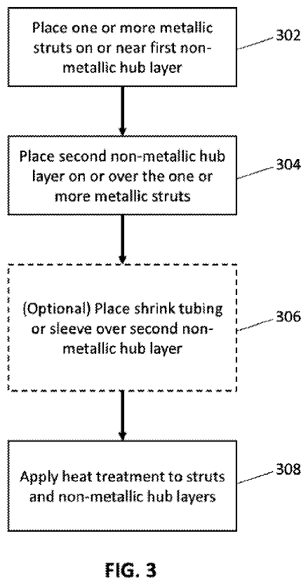

FIG. 3 is a flowchart illustrating an exemplary method of manufacturing an intravascular blood pump. At 302, the method of manufacturing can comprise placing one or more metallic struts on or near a first non-metallic hub layer. As described above, the struts can be coupled to a blood conduit of a blood pump, and can comprise a metallic material such as nitinol. The first non-metallic hub layer can comprise a non-metallic material, such as Chronoflex.

At 304, the method of manufacturing can comprise placing a second non-metallic hub layer on the one or more metallic struts. As described above, the second non-metallic hub layer can also comprise a non-metallic material, such as Pebax.

At optional step 306, shrink tubing or a sleeve can be placed over the assembly that includes the first non-metallic hub layer, the struts, and the second non-metallic hub layer. The shrink tubing can assist in providing a more uniform blending/melting of the layers as described below in step 308.

At 308, the method of manufacturing can further include applying heat treatment to the struts and non-metallic hub layers to melt or meld the layers so as to encompass, surround, or secure the struts into the hub. When the shrink tubing or sleeve of optional step 306 is included, the melting or melding of the layers can be more uniform.

Although the above examples show and describe a distal hub that is distal to the blood conduit, in some cases, a proximal hub having similar non-turbulent flow promoting features (e.g., channels) may be positioned proximal to the conduit. For example, a proximal hub may have a body and spokes shaped to form channels that promote non-turbulent flow out of a proximal opening (e.g., outlet) of the conduit. Such proximal hub may be used with or without the non-turbulent flow promoting distal hub shown.

Any of the blood pumps described herein may include surfaces with one or more anticoagulant agents. For example, at least a portion of one or more of the hubs, conduits (e.g., scaffold and/or membrane), struts (e.g., proximal and/or distal struts), distal tips and/or impellers of the blood pumps described herein may include a coating or material having an anticoagulant agent. In some cases, the anticoagulant agents may include drugs such as heparin, warfarin and/or prostaglandins.

What is claimed is:

1. An intravascular blood pump, comprising:
a collapsible conduit having an inner lumen for passing fluid therethrough, the conduit comprising a proximal end having a proximal opening, and a distal end having a distal opening;
at least one impeller within the conduit, the at least one impeller arranged to pump fluid into the distal opening of the conduit and out of the proximal opening of the conduit;
a plurality of metallic struts extending from the proximal end or the distal end of the conduit; and
a hub configured to receive the plurality of metallic struts, the hub comprising at least one non-metallic layer.

2. The pump of claim 1, wherein the hub comprises a first non-metallic layer and a second non-metallic layer disposed around the plurality of metallic struts.

3. The pump of claim 2, wherein the first non-metallic layer comprises a thermoplastic urethane.

4. The pump of claim 2, wherein the second non-metallic layer comprises Pebax.

5. The pump of claim 1, wherein the at least one non-metallic layer is configured to encompass or surround the plurality of metallic struts.

6. The pump of claim 2, wherein the first non-metallic layer and the second non-metallic layer are heat treated so as to meld together around the plurality of metallic struts.

7. The pump of claim 1, wherein the hub comprises a distal hub positioned adjacent to the distal opening.

8. The pump of claim 1, further comprising a bullet-shaped tapering section extending proximally from where the plurality of metallic struts are joined to the distal hub.

9. The pump of claim 1, wherein the plurality of metallic struts comprise nitinol.

10. The pump of claim 1, wherein the plurality of metallic struts comprises 4, 5, 6, 7, or 8 struts.

11. An intravascular blood pump, comprising:
a collapsible conduit having an inner lumen for passing fluid therethrough, the conduit comprising a proximal end having a proximal opening, and a distal end having a distal opening;
a proximal impeller positioned at least partially within the conduit near the proximal opening, the at least one impeller arranged to pump fluid into the distal opening of the conduit and out of the proximal opening of the conduit;
a plurality of metallic struts extending from the distal end of the conduit; and
a distal hub configured to receive the plurality of metallic struts, the hub comprising at least one non-metallic layer configured to encapsulate the plurality of metallic struts.

12. The pump of claim 11, wherein the hub comprises a first non-metallic layer and a second non-metallic layer disposed around the plurality of metallic struts.

13. The pump of claim 12, wherein the first non-metallic layer comprises a thermoplastic urethane.

14. The pump of claim 12, wherein the second non-metallic layer comprises Pebax.

15. The pump of claim 11, wherein the at least one non-metallic layer is configured to encompass or surround the plurality of metallic struts.

16. The pump of claim 12, wherein the first non-metallic layer and the second non-metallic layer are heat treated so as to meld together around the plurality of metallic struts.

17. The pump of claim 11, wherein the hub comprises a distal hub positioned adjacent to the distal opening.

18. The pump of claim 11, further comprising a bullet-shaped tapering section extending proximally from where the plurality of metallic struts are joined to the distal hub.

19. The pump of claim 11, wherein the plurality of metallic struts comprise nitinol.

20. The pump of claim 11, wherein the plurality of metallic struts comprises 4, 5, 6, 7, or 8 struts.

21. A method of manufacturing an intravascular blood pump, the method comprising:
placing one or more metallic struts of a blood conduit on or near a first non-metallic hub layer;
placing a second non-metallic hub layer on or over the one or more metallic struts; and
applying a heat treatment to the one or more metallic struts, the first non-metallic hub layer, and the second non-metallic hub layer to meld or melt the first and second non-metallic hub layers around the one or more metallic struts.

22. The method of claim 21, further comprising, prior to the applying a heat treatment step, applying a shrink tubing over the second non-metallic hub layer.

23. The method of claim 22, further comprising removing the shrink tubing after applying the heat treatment.
